(19) United States
(12) Plant Patent Application Publication
(10) Pub. No.: US 2006/0137052 P1
(43) Pub. Date: Jun. 22, 2006

(54) LACEBARK ELM TREE NAMED 'WHIT XXX'

(50) ULMUS PARVIFOLIA; LACEBARK ELM

(76) Inventor: Carl E. Whitcomb, Stillwater, OK (US)

Correspondence Address: STREETS & STEELE, 13831 NORTHWEST FREEWAY, SUITE 355, HOUSTON, TX, 77040, US

(21) Appl. No.: 11/020,611

(22) Filed: Dec. 22, 2004

(51) Int. Cl. A01H 5/00

(52) U.S. Cl. PLT/221

(57) ABSTRACT

A new and distinct lacebark elm tree, particularly distinguished in having an attractive upright-oval form, dark yellow-green leathery leaves and exceptional wood strength. The tree is easily propagated from cuttings to produce trees having a strong central leader and good stem taper without staking. The tree has proven resistant to common elm insects and diseases. The observed tree produces few seeds, none of which have been viable.

### GENUS AND SPECIES

Ulmus parvifolia

### VARIETAL DENOMINATION

Lacebark Elm ‘Whit XXX’

### BACKGROUND OF THE INVENTION

1. Field of the Invention

This invention relates to a new and distinct variety or cultivar of the deciduous tree Ulmus parvifolia, commonly known as the lacebark elm.

2. Description of the Related Art

Lacebark elm, Ulmus parvifolia, was originally given the common name Chinese elm. Unfortunately, this common name was confusingly similar to the common name of Ulmus pumil, the Siberian elm. Because of the frequent confusion resulting from the similarity of these common names, Mr. E. W. Johnson of the U.S. Department of Agriculture and of Woodward, Okla. began referring to Ulmus parvifolia as lace-bark elm. Mr. Johnson used the common name lace-bark elm for Ulmus parvifolia in his writings on the introduction and study of trees for windbreaks: Ornamental and Windbreak Trees for the Southern Great Plains, USDA Crops Research Bulletin ARS 34-77 (1966). In 1973, the inventor of the present new cultivar began using and promoting the common name lacebark elm in his many writings as a substitute for the less suitable name Chinese elm. The name lacebark elm is now nearly universally accepted as the common name.

The lacebark elm is native to northern and central China, extending eastward into Korea. Typically, the lacebark elm is a large tree that matures to between about forty and about fifty feet tall and about the same dimensions wide. The lacebark elm is very adaptable to poor soil conditions and may be further characterized as having leaves that are dark green and having bark that is attractive.

The original seed from which this new cultivar is a descendent was collected south of Sian, China in 1914 and sent to the United States by U.S.D.A. plant explorer Frank N. Meyer. The resulting seedlings contributed to the Prairie State Forestry Project between 1935 and 1942 and the resulting 17,000 mile shelterbelt system. According to Mr. Johnson, at least a portion of the seed sent by Meyer ended up at the U.S.D.A. Southern Great Plains Field Station in Woodward, Okla. Mr. Johnson noted in 1966 that growth of Ulmus parvifolia seedlings was quite variable and that small leaved selections were typically more cold and drought tolerant.

The original seed used to develop this new cultivar was obtained by the inventor in 1972 from a large lacebark elm tree with small, glossy leaves located at the home of Mr. E. W. Johnson in Woodward, Okla. This large lacebark elm tree was grown from the seed sent back from Sian, China in 1914. The inventor also took cuttings from Mr. Johnson's tree on three occasions, but none rooted. However, thousands of lacebark elm seedlings were grown from those original seeds collected in 1972 from Mr. Johnson's tree.

By 1984, thirteen outstanding tree seedlings had been selected from all those grown from the seeds collected in 1972. However, each of these thirteen selections responded quite differently to attempts to propagate them from cuttings. No cuttings were ever rooted from 3 of the thirteen selections and these selections were discarded. Five selections rooted poorly, generally with a success rate of less than 25%, so these selections were also discarded. The five selections that rooted the best were studied in detail by Gary G. Hickman and the inventor. Of these five, only two grew moderately well following rooting of the cuttings, but the growth form of the offsprings from these two selections required considerable pruning and training at an early age to make desirable trees resembling the parents. These selections were judged to require excessive work to develop into desirable tree forms, so these selections were also discarded.

In 1984, the inventor wrote in his book entitled Plant Production In Containers, Lacebark Publications, Stillwater, Okla. (1984, 1988, 2003): “This study leaves little doubt about great differences among individual seedlings to root and grow into useful plants. If an outstanding tree is located, the first step is to determine if it will root from cuttings. The second step is to evaluate the growth and form and quality of the rooted cuttings. If the tree fails on either evaluation, look for other trees of the same species with good qualities and continue. Just because one tree of a species does not root or grow well does not mean that another specimen will respond the same way.”

In 1988, seeds were collected from a desirable lacebark elm in Stillwater, Okla. that had resulted from the seeds collected in 1972 from the large lacebark elm tree of Mr. Johnson in Woodward, Okla. These seeds were planted, resulting in approximately sixty lacebark elm trees. The inventor then collected seeds from the two best specimens of these sixty lacebark elm trees and planted them in containers, resulting in approximately 2,000 tree seedlings. The most desirable of these seedlings, about 800, were selected and planted into the field for further evaluation. In the fall of 1996, seeds were collected by the inventor from two of these 800 trees, chosen for their demonstrated exceptional growth. The collected seeds from these two trees were planted, resulting in about 2800 seedlings. The inventor evaluated these seedlings, culling out the undesirable ones until only the best nine seedlings remained. These seedlings were characterized as having small leaves, exceptional vigor, a central leader and good form.

In February, 2002, when the nine selected trees were about 12 feet tall with a 3 inch stem diameter at the base, a severe ice storm occurred. Only one tree survived this ice storm with no damage and still standing upright, while the other eight trees were severely bent over and suffered broken limbs as a result of the ice. Then again in February, 2003 the nine trees were subjected to an even more severe ice storm and the same one tree, the new cultivar of the present invention, remained upright with no damage. One of the nine trees broke off near the ground as a result of the ice storm and the other seven trees were severely bent over and suffered broken limbs.

This new and distinct plant was asexually reproduced by rooting softwood cuttings taken from the original plant near Stillwater, Okla. The asexually reproduced plants show the unique features that characterize this new lacebark elm tree indicating that the unique features of this plant are stable through its successive generations of asexual reproduction. Furthermore, the asexually reproduced offspring grow with exceptional vigor and with a strong central leader stem unlike any before experienced by the inventor.

### SUMMARY OF THE INVENTION

The present invention resulted from the discovery of a new and distinct variety of lacebark elm tree, Ulmus parvifolia, which has been given the cultivar name ‘Whit XXX’. ‘Whit XXX’ is characterized as being a tree form with a central leader, exceptionally strong wood and developing an oval crown. Leaves are small, leathery and dark green.

### BRIEF DESCRIPTIONS OF THE DRAWINGS

FIG. 1 is a full color photographic view of my new lacebark elm tree near Stillwater, Okla.

FIG. 2 is a full color photographic view of mature leaves on a twig produced during early to mid spring.

FIG. 3 is a full color photographic view of leaves ranging from very young to being fully expanded on a young twig produced during summer on the tree shown in FIG. 1.

FIG. 4 is a full color photographic view of green young seeds, reddish purple seeds of intermediate development, and light tan mature seeds.

FIG. 5 is a full color photographic view of a section of stem about two inches diameter.

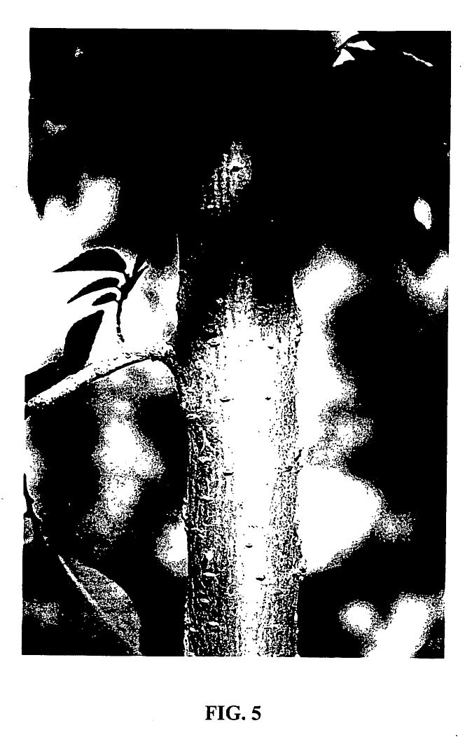

FIG. 6 is a full color photographic view of a section of stem about four inches diameter from the tree shown in FIG. 1.

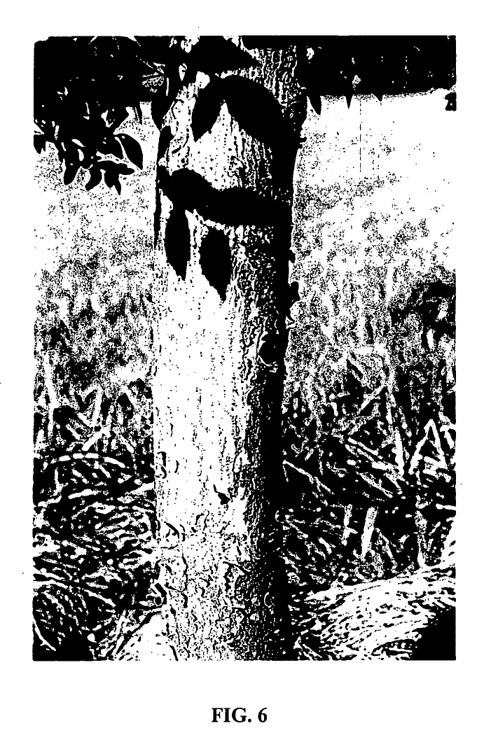

FIG. 7 is a full color photographic view comparing my new lacebark elm tree following a severe ice event with a severely ice-damaged tree of the same species, age and size.

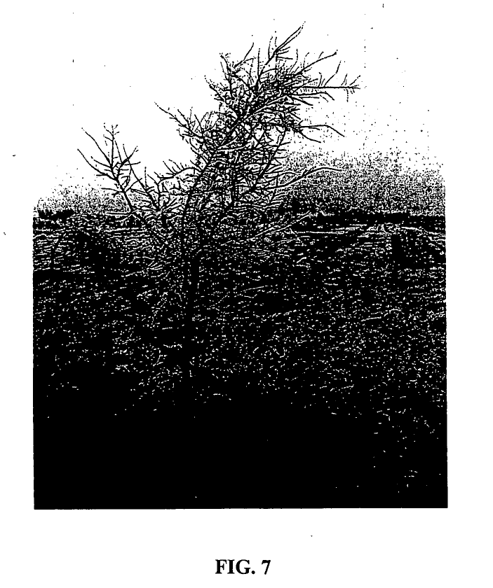

FIG. 8 is a full color photographic view of asexually reproduced offspring from my invention after three months and 15 months following rooting of cuttings.

### BOTANICAL DESCRIPTION OF THE PLANT

The following botanical description is of the new and distinct cultivar of lacebark elm tree, Ulmus parvifolia, named ‘WHIT XXX’. Specific color designations set forth by number designations are in accordance with The Royal Horticultural Society Colour Chart. General color recitations are consistent with ordinary American color terminology.

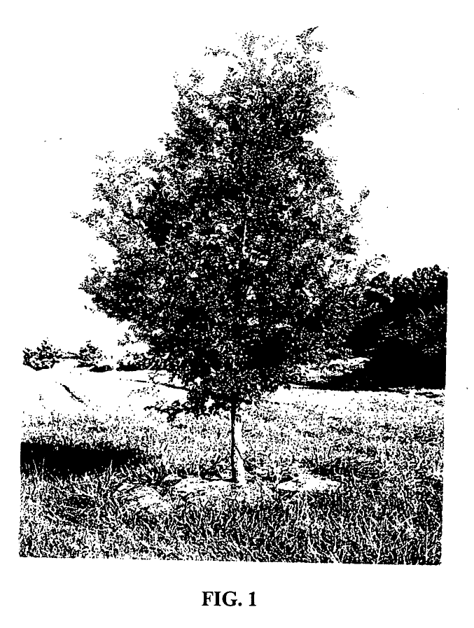

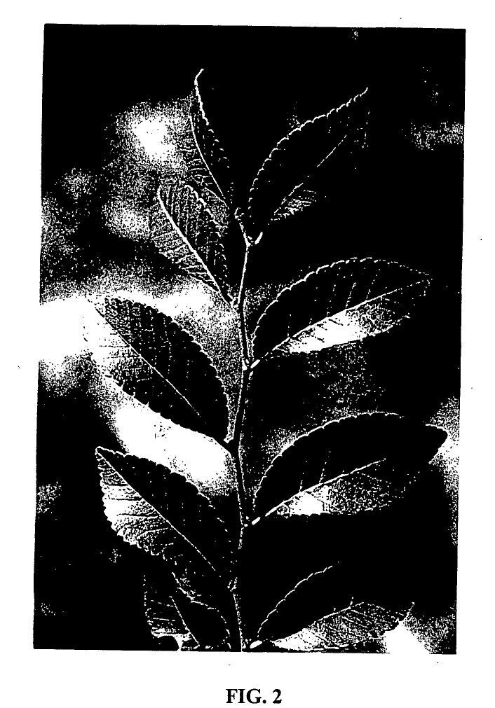

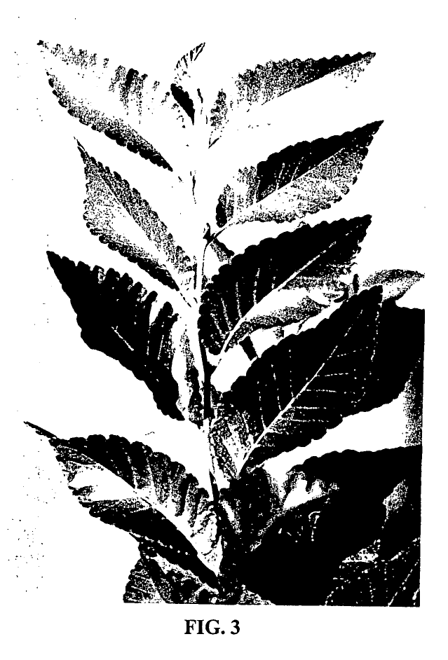

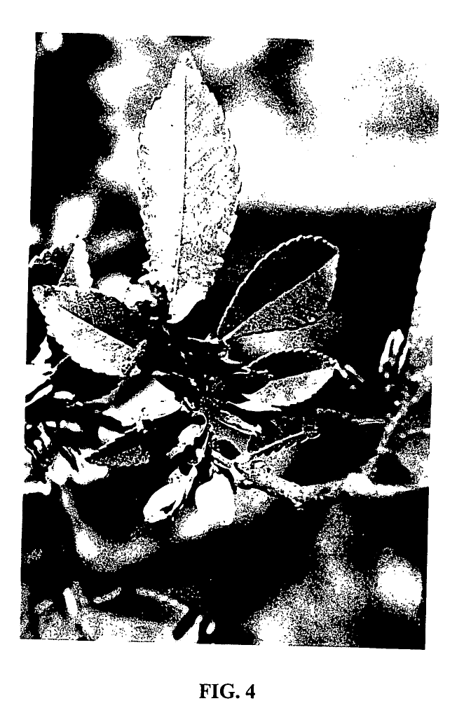

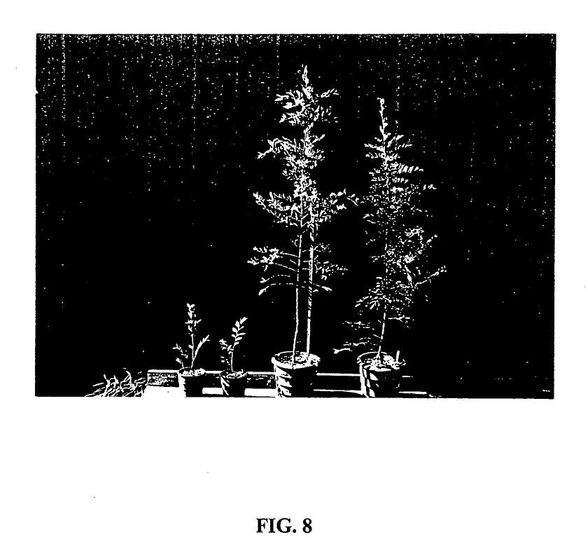

The lacebark elm ‘WHIT XXX’ has not been observed under all possible environmental conditions. It is to be understood that the phenotype may vary significantly with variations in environment such as soils, temperature, light intensity and length of day without differences in the genotype of the plant. The following botanical characteristics and observations are taken from the plant when grown under normal outdoor conditions in north central Oklahoma. The described plant was about eight years old growing in a field in north central Oklahoma.
The plant:
   Type.—Deciduous woody tree with a single or multiple stems.
   Classification.—Lacebark Elm, Ulmus parvifolia.
   Growth habit.—Strongly upright central leader when young, as shown in FIG. 8, and becoming more oval with age, as shown in FIG. 1. The apical dominance of the central leader declines with age, leading to a broad oval form.
   Origin.—An open pollinated cross in an elm selection/breeding program in Stillwater, Okla.
   Parentage.—The parent plant of my new cultivar was a fourth generation seedling taken from an unknown tree in Sian, China in 1914. The parent plant originated from seeds taken from seedlings planted in 1997, which originated from seedlings planted in 1988, which originated from seeds collected from a mature lacebark elm in Woodward, Okla. in 1972, which originated from seeds collected from an unknown tree in Sian, China. The seeds were gathered selectively from each generation of trees having the better growth habit. The lacebark elm tree in Woodward, Okla. is still growing.
   Propagation.—The plant is easy to propagate from softwood cuttings collected during May, June and July in north central Oklahoma, with the distinguishing characteristics of the asexually propagated offspring remaining identical to the parent.
   Size and Shape.—The growth habit of my new cultivar is characterized as having an oval tree form with a strong central leader during the first four to six years following propagation. Once attaining a height of between about 10 and 14 feet, control by the central leader diminishes and branches form a broader oval crown. The growth rate is vigorous in early spring through early summer and continues into midsummer when moisture is adequate. Branches are produced in abundance along the main stem when young and are typically between about 70 and 80 degrees from vertical. The branches are alternately spaced between about one and three inches apart. As the tree matures, the angles of the branches occurring in the lower five to eight feet up the central stem are between about 35 and 45 degrees. At a height of about 10 to 14 feet, the angle of the branches is between about 30 and 35 degrees. In north central Oklahoma, the plant size is characterized as having a height of between about 30 and about 50 feet.
   Hardiness.—USDA hardiness zones 5 through 9.
   General health and pest susceptibility.—The foliage of the lacebark elm ‘Whit XXX’ has remained pest free in central Oklahoma. The stems of the plant have remained free from Nectria canker.
Foliage:
   Leaf persistence.—Deciduous.
   Arrangement on stems.—Alternate.
   Shape of leaves.—As shown in FIGS. 2 and 3, the overall leaf shape is oblique, with acute leaf tips that are short and pointed. The leaf base is oblique with the two sides slightly unequal.
   Size of leaves.—Typically, the leaves are small, between about 0.75 and about 2 inches long and between about 0.5 and about 1 inch wide with spring growth. Vigorous summer shoots may produce leaves that are between about 1 and about 3 inches long and between about 0.75 and about 1.5 inches wide.
   Margins of leaves.—Serrate, occasionally doubly serrate.
   Quantity.—Singles that alternate on the twig.
   Color of leaves.—Young and intermediate aged leaves are shiny and light, yellow-green (144-A) on the upper surface and slightly lighter green (147-B or C) on the lower surface. The petioles are light green (147-C or D). Mature leaves are shiny and dark yellow-green (147-A) on the upper surface and slightly lighter (147-B) on the lower surface. The petioles are yellow-green (147-C). Mature leaves in fall remain the same color and remain attached to the twigs. Only after several light frosts do upper portions of the leaves exposed to sun turn dull purple (183-A or B or C), while portions of leaves shaded by other leaves remain the mature color. Leaf drop typically does not occur until one or more hard freezes. Fall color is more pronounced during years when temperatures drop gradually and less when a early hard freeze causes leaves to drop.
   Texture of leaves.—The leaves are leathery, with a slightly scabrous upper surface. The lower surface is glabrous but with prominent veins.
   Leaf petiole.—Short and stout, typically between about ⅛ and about ¼ inch long. Color descriptions are provided, supra.
   Stipules.—None.
   Stems.—The stem color changes with size. Young twigs are yellow-green (146-A or B or C) and scabrous. Stems having a diameter between about ¼ and about 1 inch are grey-green (197-A or B) while stems having a diameter of about 2 inches are grey (201-A or B or 197-A). Stems having a diameter of between about 3.5 inches and about 4 inches are mottled and have patches of different colors: patches of grey-green (197-A, B or C), patches of grey (201-A or B) and patches of brownish-orange (177-B, C or D). The lenticles are brownish-orange (177-A, B or C or 172-A or B).
   Dormant vegetative buds.—The buds are elongated, sharp pointed and scabrous and are located in leaf axils.
Flowers and fruits: The flowers are typical of the species, characterized as being inconspicuous and occurring in early to mid-September in north central Oklahoma. The fruit is an elliptic-ovate samara between about ¼ and about ⅜ inch. Samara, as shown in FIG. 4, is at first light-green (139-C or D), then reddish-purple (67-A, B or C) and then light tan (199-B, C or D) at maturity. Fruit production is very light compared to other trees of the species and to date, no seeds have been viable.

I claim:

1. A new and distinct variety of lacebark elm tree, substantially as illustrated and described.
